(19) United States
(12) Patent Application Publication
(10) Pub. No.: US 2026/0118316 A1
(43) Pub. Date: Apr. 30, 2026

(54) CONCRETE MOISTURE CONTENT DIAGNOSIS DEVICE AND SYSTEM, AND DIAGNOSIS METHOD USING THE SAME

(71) Applicant: Samsung Electronics Co., Ltd., Suwon-si (KR)

(72) Inventors: JAUK GU, Suwon-si (KR); CHANG HYEON KIM, Suwon-si (KR); Eun-Ho Lee, Suwon-si (KR); JI HUN KIM, Suwon-si (KR); YOUNG DAE SHIM, Suwon-si (KR); JINHOON PARK, Suwon-si (KR)

(73) Assignees: Samsung Electronics Co., Ltd., Suwon-si (KR); Research & Business Foundation SUNGKYUNKWAN UNIVERSITY, Suwon-si (KR)

(21) Appl. No.: 19/215,591

(22) Filed: May 22, 2025

(30) Foreign Application Priority Data
Oct. 31, 2024 (KR) 10-2024-0152691

(51) Int. Cl. G01N 27/60; G01N 33/38

(52) U.S. Cl. CPC G01N 27/605; G01N 33/383

(57) ABSTRACT

A device for diagnosing a moisture content of concrete includes an electromagnetic proximity sensor configured to cause an electromagnetic field to penetrate to a certain depth of concrete and to receive a detection signal that varies depending on a permittivity of the concrete within the covered region of the electromagnetic field; a current supply unit configured to supply current to the electromagnetic proximity sensor; and a diagnostic unit configured to analyze a moisture content of the concrete by using the detection signal.

### CROSS-REFERENCE TO RELATED APPLICATION

This application claims priority to and the benefit of Korean Patent Application No. 10-2024-0152691 filed in the Korean Intellectual Property Office on Oct. 31, 2024, the entire contents of which are incorporated herein by reference.

### BACKGROUND OF THE INVENTION

### (a) Field of the Invention

The present disclosure relates to a concrete moisture content diagnosis device and system, and a diagnosis method using the same.

### (b) Description of the Related Art

In the case of a structure made of concrete, the amount of moisture in the structure is a factor that affects the strength of the structure. In order to prevent poor construction, a process of checking the amount of moisture in the structure during the construction process is required. In the case of a completed structure, when the amount of moisture in the structure increases due to water leakage, the structural strength decreases, so the process of diagnosing the amount of moisture in the structure is important to ensure the long-term stability of the structure.

Among the conventional methods of diagnosing leakage of concrete structures, the method of diagnosing leakage through the outer wall of the concrete structure is a method of measuring the change in electrical resistance in concrete by contacting the leakage detection sensor with the concrete surface.

However, due to the characteristics of concrete, the sensitivity of the detection sensor is lowered, and the depth of diagnosis is not deep, and it is difficult to accurately diagnose the degree of leakage. In addition, high proficiency is generally required for diagnosis, so subjective judgment is inevitable.

As a method to increase the diagnostic depth in concrete and confirm the result of the degree of leakage in objective numbers, a method of installing a leakage sensor in advance in concrete structures can be used. However, this cannot be applied to existing finished structures.

### SUMMARY OF THE INVENTION

The present disclosure describes a concrete moisture content diagnosis device and system, and a diagnosis method using the same that are capable of diagnosing a location and the degree of leakage inside concrete with high accuracy by penetrating an electromagnetic field to a certain depth in concrete by using an electromagnetic proximity sensor and analyzing the amount of moisture in the concrete by using detection signals that differ according to a permittivity of concrete.

Further, the present disclosure describes a concrete moisture content diagnosis device and system, and a diagnosis method using the same, in which a moisture content diagnosis device includes an autonomous moving device that enables diagnosis as the moisture content diagnosis device moves along an outer wall of concrete, to make multi-faceted diagnosis easy without being affected by user proficiency and without being limited to a height of a concrete structure, and the like.

An embodiment of the present disclosure provides a device for diagnosing a moisture content of concrete, the device including: an electromagnetic proximity sensor configured to cause an electromagnetic field to penetrate to a certain depth of concrete and to receive a detection signal that varies depending on a permittivity of the concrete within the covered region of the electromagnetic field; a current supply unit configured to supply current to the electromagnetic proximity sensor; and a diagnostic unit configured to analyze a moisture content of the concrete by using the detection signal.

Another embodiment of the present disclosure provides a system for diagnosing a moisture content of concrete, the system including: a concrete moisture content diagnosis device which includes an electromagnetic proximity sensor having a first side configured to face the concrete while making an electromagnetic field penetrate into the concrete, and which analyzes a moisture content of the concrete by using a detection signal that varies according to a permittivity of the concrete; and a moving device configured to connect to a second side of the moisture content diagnosis device to move the moisture content diagnosis device.

Still another embodiment of the present disclosure provides a method of diagnosing a moisture content of concrete, the method including: making, by an electromagnetic proximity sensor, an electromagnetic field penetrate to a certain depth of concrete and receiving a detection signal that varies depending on a permittivity of the concrete; deriving a permittivity of the concrete by using the detection signal; and determining a moisture content of the concrete according to the derived permittivity.

According to the embodiments, it is possible to diagnose the location of leakage and the degree of leakage inside the concrete through the outer wall of the concrete without changing the existing concrete structure.

### BRIEF DESCRIPTION OF THE DRAWINGS

FIG. 1 is a diagram illustrating a concrete moisture content diagnosis device according to an embodiment.

FIG. 2 is a diagram illustrating a configuration of the concrete moisture content diagnosis device according to an embodiment.

FIGS. 3, 4A, and 4B are diagrams illustrating a concrete moisture content diagnosis system according to an embodiment.

FIG. 5 is a diagram illustrating a configuration of the concrete moisture content diagnosis system according to an embodiment.

FIGS. 6A, 6B, 7A, 7B, 8A, 8B, 9A, 9B, 9C, 10A, 10B, 10C, 11A, and 11B are diagrams illustrating an experiment for verifying a result of a concrete moisture content test according to the concrete moisture content diagnosis device according to an embodiment of the present disclosure.

FIGS. 12 to 15 are flowcharts illustrating a concrete moisture content diagnosis method according to an embodiment.

### DETAILED DESCRIPTION OF THE EMBODIMENTS

In the following detailed description, only certain embodiments of the present disclosure have been illustrated and described, simply by way of illustration. The present disclosure can be variously implemented and is not limited to the following embodiments.

The drawings and description are to be regarded as illustrative in nature and not restrictive. Like reference numerals designate like elements throughout the specification.

In addition, the size and thickness of each configuration illustrated in the drawings are arbitrarily illustrated for understanding and ease of description, but the present disclosure is not limited thereto. In the drawings, for understanding and ease of description, the thickness of layers, films, panels, regions, areas, etc., may be exaggerated for clarity.

Throughout the specification, when a part is said to be “connected” to another part, this includes not only a case where the parts are “directly connected”, but also a case where the parts are “indirectly connected” with another member interposed therebetween. In addition, unless explicitly described to the contrary, the word “comprise”, and variations such as “comprises” or “comprising”, will be understood to imply the inclusion of stated elements but not the exclusion of any other elements.

Further, it will be understood that when an element such as a layer, film, region, or substrate is referred to as being “on” another element, it may be directly on the other element or intervening elements may also be present. In contrast, when an element is referred to as being “directly on” another element, or as “contacting” another element, there are no intervening elements present at the point of contact. when an element is “on” a reference portion, the element is located above or below the reference portion, and it does not necessarily mean that the element is located “above” or “on” in a direction opposite to gravity.

Further, in the entire specification, as the term “in a plan view”, refers to a view in which a target part is viewed from above, and as the term “in a cross-sectional view”, refers to a view in which the cross-section is obtained by cutting a target part vertically and viewing from the side.

The various “units” described herein for performing various functions may be implemented using a processor (i.e., a hardware circuit), such as a microprocessor, a CPU (Central Processing Unit), a GPU (graphics processor), a digital signal processor (DSP), a field-programmable gate array (FPGA), etc., along with a memory, and may be part of a computer. Such units may be formed by several interconnected processors or controllers and may be configured by software.

Hereinafter, a concrete moisture content diagnosis device 100, a system 10, and a diagnosis method using the same according to an embodiment of the present disclosure will be described in more detail with reference to the drawings.

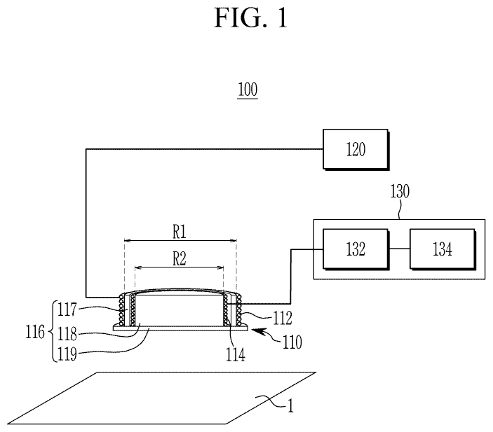

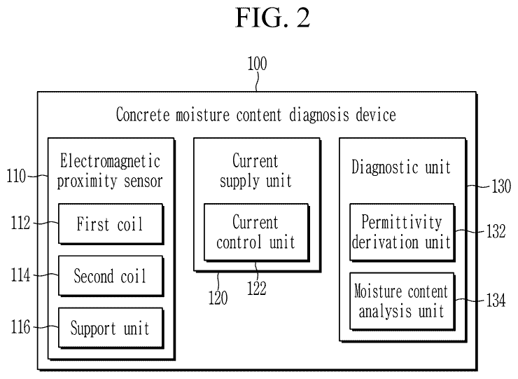

FIG. 1 is a diagram illustrating a concrete moisture content diagnosis device according to an embodiment, and FIG. 2 is a diagram illustrating a configuration of the concrete moisture content diagnosis device according to the embodiment of FIG. 1.

The concrete moisture content diagnosis device 100 according to the present disclosure may be a device for diagnosing a leak in the concrete 1. Since the concrete 1 is a non-conductive material, the diagnostic principle using an electromagnetic field is as follows.

First, the electromagnetic field (primary electric field) generated in a first coil 112 of an electromagnetic proximity sensor 110 may induce a displacement current in the electromagnetic proximity sensor 110 depending on the permittivity of the concrete 1. An alternating current may be supplied to the first coil 112.

Due to this displacement current, an induced magnetic field (secondary magnetic field) may be generated, and the induced magnetic field may affect the induced current induced in the second coil 114 of the electromagnetic proximity sensor 110.

The moisture content diagnosis device 100 according to the present disclosure may diagnose the degree of leakage of the concrete 1, for example, the moisture content, by using the induced current induced in the second coil 114.

The induced current depends on the moisture content of the concrete 1, and an impedance value of the second coil 114 through which the induced current flows may be measured to analyze the degree of leakage of the concrete 1 based on the measured impedance value.

As shown in FIGS. 1 and 2, the moisture content diagnosis device 100 according to the present disclosure may include the electromagnetic proximity sensor 110, which makes an electromagnetic field penetrate to a predetermined depth of the concrete 1 and receives a detection signal that differs, or varies, according to the permittivity of the concrete 1, a current supply unit 120 that supplies a current to the electromagnetic proximity sensor 110, and a diagnostic unit 130 that analyzes the moisture content of the concrete 1 by using the detection signal.

The electromagnetic proximity sensor 110 may analyze the moisture content of the concrete 1 in a non-contact state with the concrete 1.

The diagnostic unit 130 may include one or more circuits connected to one or more processors, which may be operated according to software such as computer program code, configured to determine various information based on a received detection signal. For example, the diagnostic unit 130 may include a permittivity derivation unit 132 that derives the permittivity of the concrete 1 by using the detection signal received through the electromagnetic proximity sensor 110, and a moisture content analysis unit 134 that analyzes the moisture content of the concrete 1 according to the derived permittivity. Each of the permittivity derivation unit 132 and moisture content analysis unit 134 may include one or more circuits connected to one or more processors, which may be operated according to software such as computer program code, configured perform analysis of permittivity and moisture content respectively. The permittivity derivation unit 132 and moisture content analysis unit 134 may be part of a single module, or may be separate modules that communicate with each other.

Here, the detection signal, which is a current induced by an electromagnetic field, is a signal for an induced current induced in the second coil 114. Specifically, the detection signal may be an impedance value for an induced current in the second coil 114.

As described above, the electromagnetic field is a primary magnetic field, which may be a magnetic field generated by an alternating current flowing through the first coil 112.

The induced current may be a current induced in the second coil 114.

As described above, the electromagnetic field (primary electric field) induces a displacement current in the concrete 1 according to the permittivity of the concrete 1, and an induced magnetic field (secondary magnetic field) is generated according to the displacement current. The generated induced magnetic field affects the induced current induced in the second coil 114.

The concrete moisture content diagnosis device 100 according to the present disclosure may analyze the moisture content of the concrete 1 by using the impedance and the permittivity of the concrete 1.

In order to verify the relationship between impedance, permittivity, and moisture content, which is the premise of the present disclosure, a verification experiment was conducted, in which the impedance, permittivity, and moisture content of the concrete 1 were obtained by the following method.

### 1) Impedance Value Analysis Method

An electromagnetic field is generated by the current supplied to the first coil 112 of the electromagnetic proximity sensor 110, the electromagnetic field induces a displacement current inside the concrete 1 according to the permittivity of the concrete 1, and an induced magnetic field is generated by the displacement current.

In this case, an induced current is generated in the second coil 114, and the induced magnetic field affects the induced current.

For impedance analysis, an impedance value (detection signal) in the second coil 114 through which the induced current flows may be analyzed. The induced current may be different depending on the moisture content of the concrete 1. Accordingly, the impedance value of the induced current may also be different.

### 2) Permittivity Derivation Method

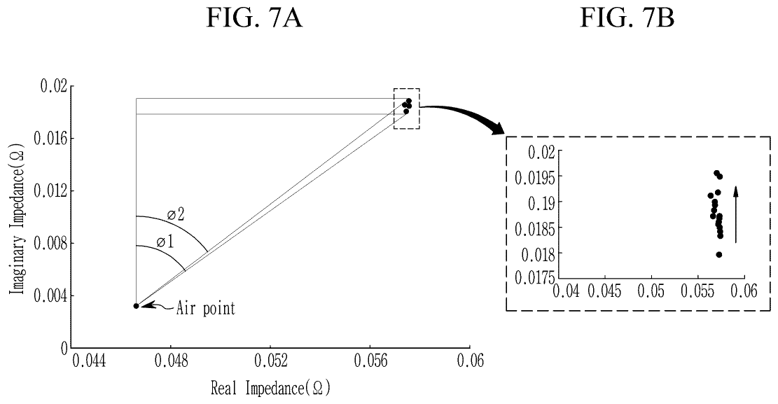

The permittivity of the concrete 1 may be derived using the analyzed impedance value. A method of deriving the permittivity of the concrete 1 from the impedance value may be derived by using a permittivity-impedance model obtained through an experiment in advance (refer to FIGS. 7 to 9).

3) Moisture Content Analysis Method

The process of analyzing the moisture content from the derived permittivity, according to one embodiment, is as follows.

Since the permittivity of the concrete 1 changes according to the moisture content, the moisture content of the concrete 1 corresponding to the permittivity may be analyzed by using the foregoing derived permittivity value.

The moisture content of the concrete 1 means the amount of moisture contained in the concrete 1. The moisture content of the concrete 1 in the experiment for verification according to one embodiment was obtained in the following manner.

With respect to the prepared concrete 1 specimen, the concrete moisture content diagnosis device 100 may be disposed on one side. Moisture (water) is disposed on the other side so that moisture moves into concrete 1 through the other side.

In this case, a distance by which moisture is absorbed into the concrete 1 and moves, that is, a distance by which moisture has moved from the other side to one side (e.g., from a first side to a second side opposite the first side), may be assumed to be the moisture height. The weight of the concrete 1 varies according to the moisture height.

The weight of the concrete 1 different according to the moisture height may be calculated, and by using this, the moisture height may be replaced by a moisture content. In this manner, in this experiment, different moisture contents were used by varying the weight of the concrete 1 in different testing examples.

In the above, the method for verification has been described, and below, the process of analyzing the moisture content of the concrete 1 by using the concrete moisture content diagnosis device 100 according to the present disclosure will be described in detail.

The electromagnetic field is generated in the concrete 1 by the electromagnetic proximity sensor 110 of the moisture content diagnosis device 100, and the process of deriving the change in the moisture content in the concrete 1 by using the change in the electromagnetic field is as follows.

FIG. 1 illustrates the moisture content diagnosis device 100, and is a diagram illustrating a state in which the electromagnetic proximity sensor 110 is cut so that the cross section thereof is seen. As illustrated in FIG. 1, the electromagnetic proximity sensor 110 has a shape wound to have a first diameter R1, and may include the first coil 112 through which a current supplied from the current supply unit 120 flows.

In addition, the electromagnetic proximity sensor 110 has a shape wound to have a second diameter R2 smaller than the first diameter R1, and may include the second coil 114 through which an induced current flows. The second coil 114 is placed apart from the inside of the first coil 112 The current supply unit 120 supplies a current to the first coil 112, and an electromagnetic field (primary magnetic field) is generated around the first coil 112 through which the current flows. The electromagnetic field may penetrate into the concrete 1.

The electromagnetic field penetrating into the concrete 1 may induce a displacement current inside the concrete 1 according to the permittivity of the concrete 1. An induced magnetic field (secondary magnetic field) may be induced by this displacement current. The induced magnetic field induced by the displacement current may affect the second coil 114. For example, the induced magnetic field induced by the displacement current may affect the induced current induced in the second coil 114.

The induced magnetic field may be different depending on the moisture content of the concrete 1. For example, an induced magnetic field induced in the second coil 114 when no moisture is present in the concrete 1 may be a certain amount, and the induced magnetic field induced in the second coil 114 when different amounts of moisture are present in the concrete 1 may result in different respective amounts that vary based on the amount of moisture in the concrete 1 (e.g., in a particular portion of the concrete covered by the primary magnetic field). As a result, the induced current induced in the second coil 114 depends on the degree of moisture content inside the concrete 1.

The electromagnetic proximity sensor 110 may derive a permittivity change for each area of the concrete 1 by analyzing a change in the induced current generated through the concrete 1.

As described above, analyzing the change in the induced current may include analyzing the impedance value of the induced current. By using a model defining the relationship between impedance and permittivity, it is possible to derive a change in permittivity from the change in impedance.

When the phase relationship of the current and the voltage in the second coil 114 changes according to the change in the impedance, the impedance value may be obtained by using the change in the phase of the current and the voltage. In addition, the difference in permittivity in the concrete 1 may be analyzed by using the change in the phase of the current and the voltage.

In this way, the moisture content diagnosis device 100 is characterized in obtaining an impedance value from an induced current to obtain a permittivity and a moisture content. By using the difference in permittivity derived in this way, the difference in moisture content of the concrete 1 can be analyzed.

Referring to FIG. 1, the electromagnetic proximity sensor 110 may include the first coil 112, the second coil 114, and the support unit 116 supporting the shape of the first coil 112 and the second coil 114.

The support unit 116 may be a support structure that includes a first support unit 117 and a second support unit 118.

The first support unit 117 may have a hollow cylindrical shape, and may have a structure in which the first coil 112 is wound around the exterior side thereof. The first support unit 117 may be a structure such as a support cylinder or support block having some other shape.

The second support unit 118 be a support structure inserted into the first support unit 117, and may have a structure in which the second coil 114 is wound around the exterior side thereof. The second support unit 118 may be a support cylinder, which may be hollow or solid. The first support unit 117, and second support unit 118 may each be formed of a core material.

The support unit 116 may further include a plate 119 having a diameter larger than the first diameter R1 and connected to one side of the first support unit 117 and the second support unit 118 to support the shape of the electromagnetic proximity sensor 110.

The plate 119 may serve to fix positions of the first support unit 117 and the second support unit 118.

The current supply unit 120 may be a power source that supplies a current (e.g., alternating current) having a high frequency.

The concrete moisture content diagnosis device 100 may further include a current control unit 122 for controlling the supplied current. The current control unit 122 may be a controller, for example, a physical dial and/or electrically-controlled circuit, to control the supplied current.

The current control unit 122 may control the intensity of the electromagnetic field (primary magnetic field) generated by the first coil 112.

The value of the frequency used by the electromagnetic proximity sensor 110 does not affect the depth at which the electromagnetic field penetrates into the concrete 1. Accordingly, the electromagnetic proximity sensor 110 according to the present disclosure may use a value with a constantly fixed frequency.

In the moisture content diagnosis device 100 according to the present disclosure, it is preferable to use a high frequency of 10 MHz or more. This is because by using a high frequency, the sensitivity due to the permittivity of the material of the concrete 1 can be increased.

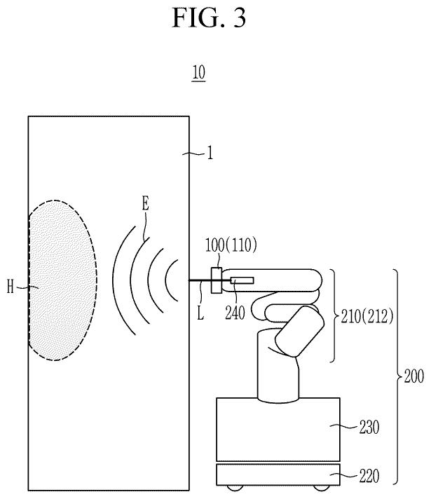

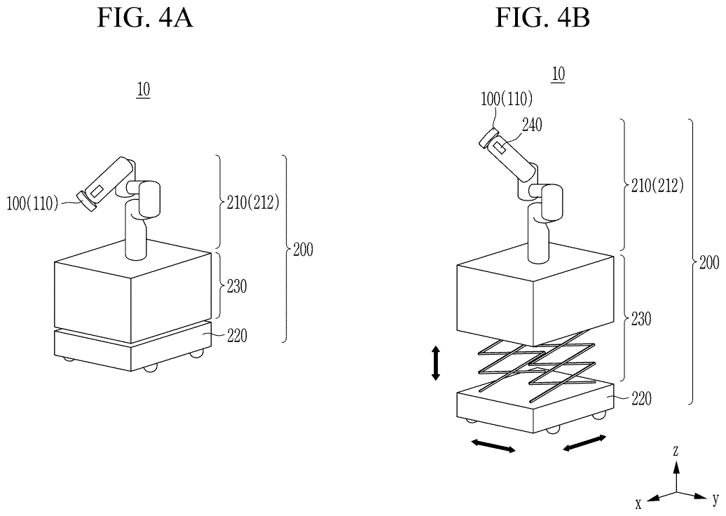

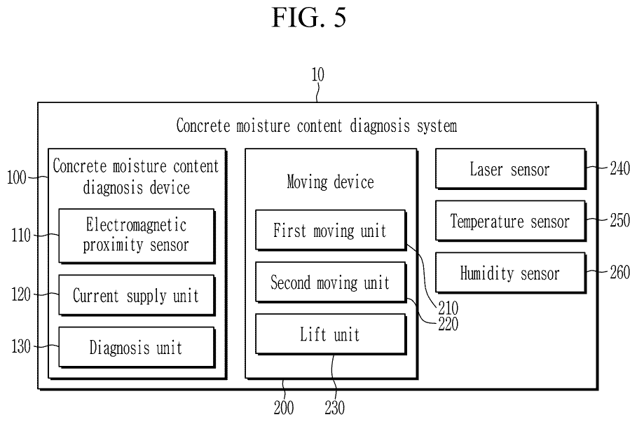

FIGS. 3 and 4 are diagrams illustrating the concrete moisture content diagnosis system according to one embodiment, and FIG. 5 is a diagram illustrating a configuration of the concrete moisture content diagnosis system according to an embodiment.

As illustrated in FIGS. 3 and 4, the concrete moisture content diagnosis system 10 according to the present disclosure may include the concrete moisture content diagnosis device 100 including the electromagnetic proximity sensor 110 and a moving device 200 connected to the other side of the moisture content diagnosis device 100 to move the moisture content diagnosis device 100.

The electromagnetic proximity sensor 110 is disposed so that one side thereof faces the concrete 1, and serves to make the electromagnetic field E penetrate into the concrete 1.

The concrete moisture content diagnosis device 100 may analyze the moisture content of the concrete 1 by using a detection signal that differs according to the permittivity of the concrete 1.

FIG. 3 shows moisture H in the concrete 1, and the electromagnetic field E penetrating toward the area containing the moisture H.

The moving device 200 may include a first moving unit 210 connected to the other side of the moisture content diagnosis device 100, and a second moving unit 220 moving in a horizontal direction while supporting the first moving unit 210.

As illustrated in FIG. 4B, the concrete moisture content diagnosis device 100 according to the present disclosure may move in all x-axis, y-axis, and z-axis directions according to the movement of the moving device 200. The first moving unit 210 may include an arm including one or more segments connected at joints to allow for movement in all directions, and the second moving unit 210 may be a mobile base, for example on wheels that allow for movement in any horizontal direction.

FIG. 4B illustrates an embodiment in which the second moving unit 220 moves in the x-axis and y-axis directions, but the moving directions are not limited thereto.

The moving device 200 may include a robot capable of autonomously driving. Depending on the embodiment, the moving device 200 may be an autonomous mobile robot (AMR).

The AMR refers to a robot having the ability to find a path by itself and move to a destination without user intervention. It is significant in that the AMR plans, moves, and performs tasks by itself, not by following a preset path.

Although not illustrated in the drawing, the moving device 200 may further include multiple sensors for autonomous driving, and may further include a path planning unit for planning a real-time path. For example, the path planning unit may include various sensors, such as geographic location sensors, visual sensors, etc., connected to a controller.

As the example, AMR may be equipped with a technology that combines functions of a LIDAR, radar RADAR, ultrasonic sensor, laser obstacle sensor, and radio frequency identification (RFID). The AMR may also include technology for communications, such as antennas, transmitters, and receivers, for wireless communications with an external device such as a computer.

As illustrated in FIGS. 3 and 4, the first moving unit 210 may have a form of an articulated arm 212 having a plurality of segments and joints connecting the segments.

Each segment and each joint of the multi-joint arms 212 may have a structure having an axis. According to the embodiment, each segment constituting the articulated arm may have a central axis and each joint may have a rotational axis. In this case, the articulated arm can rotate with respect to each axis.

For example, the first moving unit 210 may be formed of six joints and may have a structure having a total of six rotational axes as each joint includes an axis. However, the number of axes is not limited to six.

Since the first moving unit 210 has the articulated arm structure, the first moving unit 210 may move the moisture content diagnosis device 100, in particular, the electromagnetic proximity sensor 110, at various angles up and down, left and right. In addition, since the first moving unit 210 is rotatale along a plurality of axes, it is possible to perform fine angle adjustment that is not achieved by an articulated arm structure having fewer or no joints.

The moving device 200 may further include a lift unit 230 disposed between the first moving unit 210 and the second moving unit 220. The lift unit 230 may be lift that includes a platform and an extender, that cause the first moving unit 210 to be controlled to be further away from or closer to the second moving unit 220. For example, the lift unit 230 may include an actuator-controlled extender (e.g., controlled through pneumatics, hydraulics, and/or motors).

The lift unit 230 may move the first moving unit 210 in a vertical direction.

FIG. 3 and FIG. 4A are diagrams illustrating the lift unit 230 in a lowered state, and FIG. 4B is a diagram illustrating the lift unit 230 in a raised state.

The moving device 200 may further include a laser sensor 240 for emitting a laser. The laser sensor 240 may emit the laser L to a region including the concrete 1 through which the electromagnetic field E penetrates.

Referring to FIG. 3, it can be seen that the electromagnetic field E penetrates toward moisture in the concrete 1, and the laser L is emitted to the area of the concrete 1 through which the electromagnetic field E is penetrating.

The electromagnetic proximity sensor 110 according to the present disclosure diagnoses a moisture content in a non-contact state with the outer wall of the concrete 1, and may be spaced apart from the outer wall of the concrete 1 by a predetermined distance. The laser sensor 240 serves to measure the separation distance in order to correct an error that may occur due to the above-described separation distance.

In FIGS. 3 and 4, an example in which the laser sensor 240 is disposed on the first moving unit 210 is illustrated. The laser sensor 240 is for measuring the distance between the electromagnetic proximity sensor 110 and the outer wall of the concrete 1, and is preferably disposed close to the electromagnetic proximity sensor 110.

Referring to FIG. 5, the moving device 200 may further include a temperature sensor 250 that measures the temperature around the concrete 1 (e.g., at or adjacent to a surface of the concrete), and a humidity sensor 260 that measures the humidity around the concrete 1 (e.g., at or adjacent to a surface of the concrete).

In the process of measuring the moisture content of the concrete 1, the strength of the magnetic field may change according to changes in ambient temperature and humidity. Accordingly, it is important to measure the temperature and humidity around the concrete 1 to be diagnosed and correct the intensity of the magnetic field by the value of the magnetic field that changes according to temperature and humidity changes.

Accordingly, the temperature sensor 250 and the humidity sensor 260 may be considered to measure the temperature and humidity in order to correct the electrical resistance value that changes according to changes in the temperature and humidity.

The temperature sensor 250 and the humidity sensor 260 may measure the temperature and humidity around the concrete 1, and the disposed position may be freer than the disposed position of the laser sensor 240. For example, the disposed position may be in different locations on the moving device, such as on the base, on one of the arms, etc.

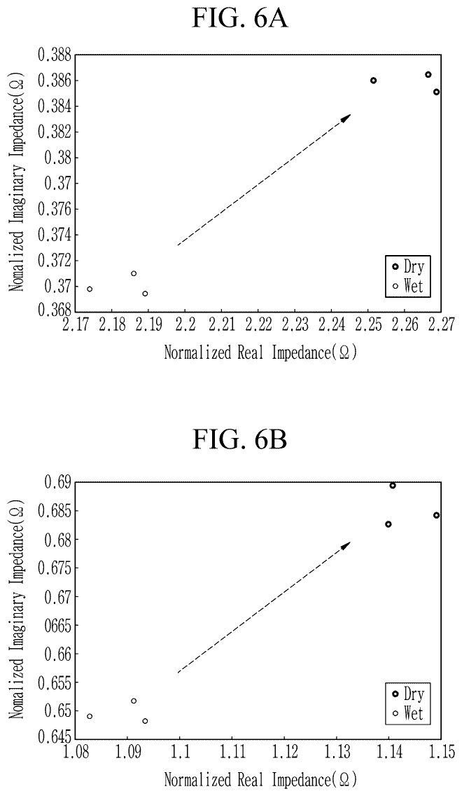

FIGS. 6 to 11 are diagrams illustrating an experiment for verifying a result of a concrete moisture content test according to the concrete moisture content diagnosis device according to an embodiment of the present disclosure.

As described above, the weight of the concrete 1 varies according to the height of the moisture penetrated into the concrete 1. The height of the moisture may be calculated by using the difference in weight of the concrete 1, and this may be replaced by the moisture content of the concrete 1.

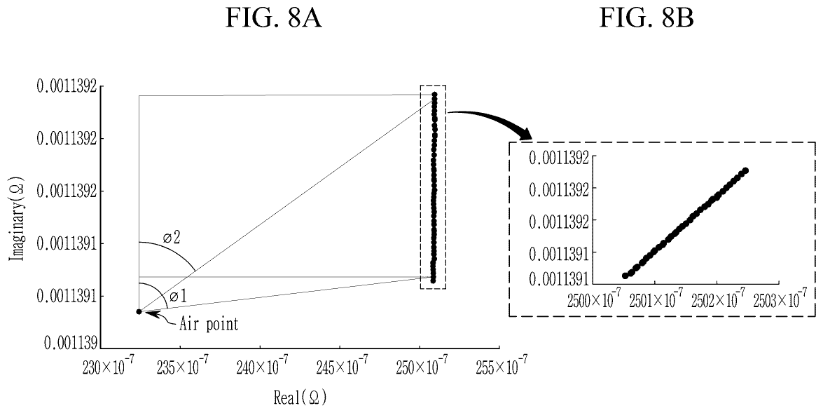

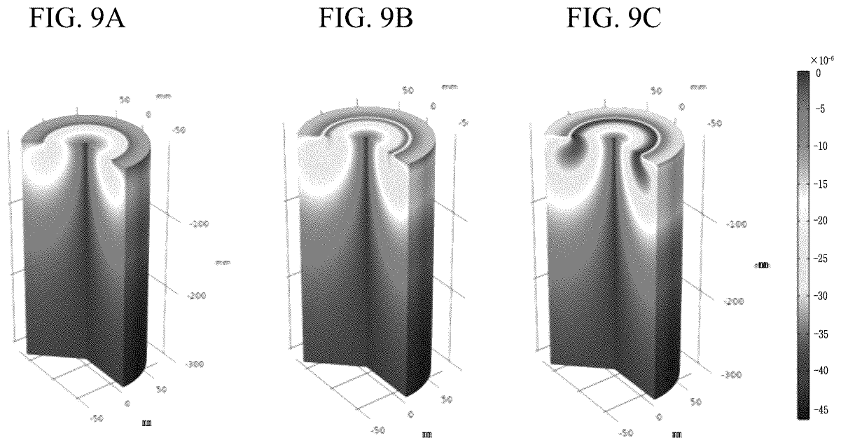

In order to match the impedance value to the permittivity, the relationship between impedance and permittivity was analyzed by using electromagnetic simulation, and a permittivity-impedance model could be obtained (see FIGS. 8 and 9).

As a result, after analyzing the impedance signal, the permittivity may be derived according to the permittivity-impedance model.

Next, the moisture content can be calculated through the derived permittivity.

Since the permittivity of the concrete 1 changes according to the moisture content, a corresponding moisture content may be obtained by using the derived permittivity.

FIGS. 6 to 9 are graphs for describing an experimental process of detecting a moisture content by using an electromagnetic field.

When moisture penetrates into the concrete 1, the electromagnetic field changes according to the height of the penetrated moisture, and FIG. 6 corresponds to a graph of the measured impedance value that changes according to the change of the electromagnetic field.

The experimental process that derived the results of FIGS. 6 to 9 is as follows.

First, moisture gradually penetrated into the concrete 1. When the electromagnetic proximity sensor 110 is disposed close to the surface of one side of the concrete 1, moisture is supplied to the other side opposite to the surface of the concrete 1, and moisture is set to penetrate therefrom.

FIG. 6A is an experiment performed with a first specimen, and FIG. 6B is an experimental value performed with a second specimen.

The depth (height) of each concrete 1 specimen was 300 mm, the electromagnetic proximity sensor 110 used a high frequency of 10 MHz, and the first diameter R1 on which the first coil 112 was wound was 100 mm.

For each specimen, impedance according to an electromagnetic field was measured according to a dry state and a wet state. A state before moisture penetrates into the concrete 1 is a dry state, and a state measured after the start of flooding is a wet state.

In a dry state and a wet state, a detection signal (induced current) was received from the second coil 114 of the electromagnetic proximity sensor 110, respectively. The points displayed on the graph are respectively measured impedance values in the induced current flowing through the second coil 114.

The process of measuring the impedance value at each point was repeated three times. On the graph, three impedance values for a specimen in a dry state and three impedance values for a specimen in a state of the lowest moisture penetration depth (wet) (a state in which moisture penetrated only into an area having the penetration depth of about 300 mm from the position where the electromagnetic proximity sensor 110 is disposed) are illustrated.

Comparing the impedance results for the first specimen and the second specimen, it may be seen that the real number of the impedance and the imaginary number value all tend to increase in the specimen in the wet state compared to the specimen in the dry state.

Impedance refers to a value that interferes with the flow of an alternating current signal, and the unit is expressed using ohms (Ω) and the alphabet Z. Impedance can be expressed in complex numbers (Z=R+jX). It is described as a real number part R and an imaginary number part X, and the real number part is called resistance, and the imaginary number part is called reactance.

In the graph representing the impedance, the X-axis represents the real number part and corresponds to a resistor that interferes with the current flow (a component that limits the current applied to the circuit). The Y-axis represents the imaginary number part and represents the reactance (a component that the circuit reacts to when an alternating current flows) that is another resistor.

Referring to the graph, the wet state (Wet) is an experimental value for a specimen with the lowest moisture penetration depth (Wet), and moisture penetrates only into an area in which the penetration depth is about 300 mm from the position where the electromagnetic proximity sensor 110 is disposed. For example, in this state, the the moisture may penetrate only a certain distance in to the concrete (e.g., a penetration depth, which may be small depth such as only 10% or 20% of the thickness of the concrete), and the distance between where the electromagnetic proximity sensor 110 is disposed and closest location to the electromagnetic proximity sensor 110 where moisture has penetrated may be about 300 mm. Even in this state, it can be seen that the measured impedance value changes.

This means that through the concrete moisture content diagnosis device 100 according to the present disclosure, it is possible to diagnose the moisture content in a portion of concrete with a depth of about 300 mm, even if the moisture only penetrates a small amount of the entire depth.

FIGS. 7A and 7B are experimental data on the premise of the same experiment as in FIG. 6.

The difference between these examples is that only the impedance values of the specimen when the moisture penetration depth is the lowest in FIG. 6 are displayed on the graph, but FIG. 7A, and 7B show all the experimental values while increasing the moisture penetration depth (i.e., the height of moisture).

FIG. 7A is a graph illustrating experimental results according to the height of moisture penetration, and FIG. 7B is an enlarged graph of a part of FIG. 7A.

The air point in FIG. 7A corresponds to a measurement value when there is nothing near the electromagnetic proximity sensor 110.

Referring to FIG. 7B, the impedance result of the concrete 1 specimen according to the moisture height may be confirmed from a plurality of data values. Specifically, it can be confirmed that when the height of moisture changes, the impedance signal changes. In particular, it can be seen that the imaginary number part of the impedance is greatly changing (moisture content=K×Φ).

FIGS. 8A and 8B are graphs illustrating electromagnetic simulation impedance results according to the permittivity of the concrete 1.

FIG. 8B is a graph illustrating an enlarged part of FIG. 8A, which corresponds to an electromagnetic simulation result according to permittivity. Here, the permittivity (εᵣ) may have a value of 1 to 50.

Since the permittivity increases as the moisture content in the concrete 1 increases, the graph is the result of the analysis of the impedance data according to the permittivity obtained by changing the permittivity of the concrete 1. In the case of actual concrete 1, the permittivity (εᵣ) has a value of 5 to 20 depending on the moisture content.

FIGS. 9A-9C show the simulation results, and FIG. 9A is the simulation result of the case where the permittivity (εᵣ) is 10, FIG. 9B is the case where the permittivity (Er) is 30, and FIG. 9C is the case where the permittivity (εᵣ) is 50.

Referring to FIGS. 8B and 9, it can be seen that the imaginary number part tends to increase as the permittivity increases.

Referring to the experimental graphs illustrated in FIGS. 7A, 7B, 8A, and 8B, each relationship may be connected through a phase value obtained by substituting a real number part value and an imaginary number part value of impedance based on an air point. Each coefficient in the equation according to the present disclosure corresponds to a value obtained through repeated experiments.

The experimental results of FIGS. 7A, 7B, 8A, and 8B are represented by using the phase (Φ) value in order to represent the corresponding real number part and imaginary number part values as one impedance data according to the moisture content. The phase (Φ) may be represented by Φ=arctan (ΔReal/Δlmaginary). Here, Δ means a difference between the air point value and the measurement signal, and the reason for using the value of Δ is to find out the phase difference of the delayed signal compared to the air point.

Referring to the experimental graphs (FIGS. 7A, 7B, 8A, and 8B) and the simulation results (FIG. 9), it can be seen that the phase (Φ) decreases as the permittivity increases. The experiment shows the impedance value according to the moisture content, but the factor that affects the actual experiment corresponds to the change in the permittivity of concrete according to the moisture content.

Since the tendency has linearity, the phase (Φ) may be expressed as a function according to the permittivity. The permittivity (εᵣ) may be expressed in k×Φ.

In the permittivity and moisture content described above, the difference in coefficients K and k in each equation may be seen as expressing the difference that occurs when each experiment is confirmed by simulation.

As a result, by using the electromagnetic proximity sensor 110 of the concrete moisture content diagnosis device 100 according to the present disclosure, the moisture content or permittivity inside the concrete 1 may be predicted by using the impedance value (phase). Therefore, the leakage state may be diagnosed.

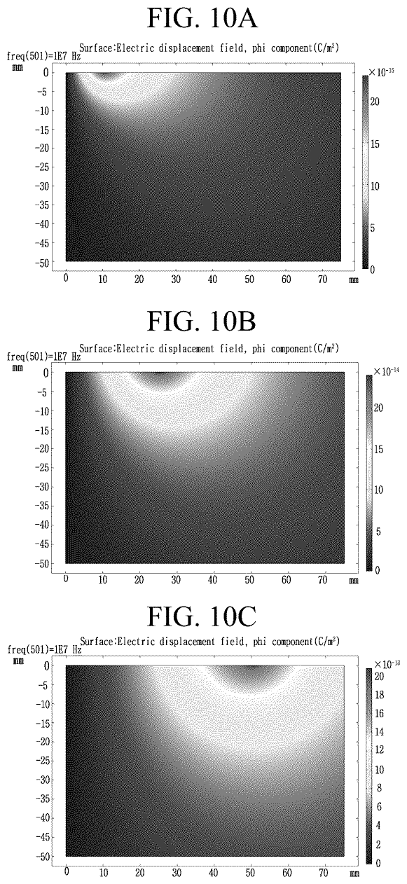

FIG. 10A-10C are graphs illustrating a change in a displacement current field in the concrete 1 according to a change in the first diameter R1 in which the first coil 112 is disposed in the electromagnetic proximity sensor 110. In this case, the frequency is constantly fixed.

Specifically, the first graph in FIG. 10A uses the electromagnetic proximity sensor 110 in which the first coil 112 is disposed so that the first diameter R1 is 20 mm, and the horizontal and vertical axes on the graph represent the cross-sectional length of the concrete 1, respectively.

First, referring to the vertical axis, the top end is the surface of the concrete 1 (i.e., the outer wall of the concrete 1 in which the electromagnetic proximity sensor 110 is disposed close), and corresponds to a region in which the depth of the concrete 1 increases toward the lower end. The vertical axis may be seen as a depth through which the electromagnetic field penetrates, for example the depth of the concrete 1.

The horizontal axis corresponds to an area range (area) of an electromagnetic field generated by the electromagnetic proximity sensor 110.

The horizontal axis may represent a cross section perpendicular to the penetration depth (vertical axis) of the electromagnetic field, and may be seen as a value representing the area of the electromagnetic field generated parallel to the surface of the concrete 1. Therefore, a covered region of the electromagnetic field may include the locations where the electromagnetic field exists along the penetration depths and areas of the electromagnetic field depicted in FIG. 10A.

The length corresponding to the horizontal axis may be any one length forming an area, and the length may be proportional to the area of the generated electromagnetic field. For example, as shown in FIG. 10A, at a particular depth within the concrete 1 and for a particular area, the electromagnetic field will have a particular magnitude, corresponding to the light to dark gradient.

The scale shown on the right side of each figure may be interpreted as representing the strength of the electromagnetic field, which indicates the detection range of the moisture content (displacement current field) within the concrete 1. That is, it represents the strength of the electromagnetic field that has penetrated into the concrete 1, where the field is strongest near the surface of the concrete 1 and gradually weakens as the depth increases.

The concrete moisture content diagnosis device 100 may measure the moisture content of the concrete 1 in a state positioned close to the surface of the concrete 1 so that the parallel surface of one side of the electromagnetic proximity sensor 110 is disposed parallel to the surface of the concrete 1. This is to further increase the diagnostic accuracy.

In this case, the electromagnetic field generated on the parallel surface of one side of the electromagnetic proximity sensor 110 reaches the surface of the concrete 1 first as soon as it occurs. It can be seen that when the surface of the concrete 1 is first reached, the electromagnetic field is generated over the widest area (i.e., the generated area). As the depth in the concrete 1 increases, the area of the electromagnetic field capable of reaching the depth gradually narrows.

Referring to the first graph in FIG. 10A, it can be seen that the electromagnetic field has reached a large area on the surface of the concrete 1, and as the depth of the concrete 1 increases, the area of the reached electromagnetic field is narrowing.

In the second graph in FIG. 10B, the electromagnetic proximity sensor 110 in which the first coil 112 is disposed so that the first diameter R1 is 50 mm is used, and in the third graph in FIG. 10C, the electromagnetic proximity sensor 110 in which the first coil 112 is disposed so that the first diameter R1 is 80 mm is used.

Referring to the three graphs in FIG. 10A to FIG. 10C, it can be seen that as the first diameter R1 on which the first coil 112 is wound increases, the depth and area of the electromagnetic field reaching the concrete 1 increase.

Therefore, referring to FIG. 10, it can be seen that as the first diameter R1 in which the first coil 112 is disposed in the electromagnetic proximity sensor 110 increases, the arrival range (depth and area) of the electromagnetic field in the concrete 1 increases. This means that a detection range (displacement current field) of a change in permittivity due to the electromagnetic field increases.

According to an embodiment, the first diameter R1 at which the first coil 112 is disposed may be 80 mm or more.

Through experiments, it was confirmed that the electromagnetic field could penetrate up to 300 mm in the concrete 1 when the first diameter R1 where the first coil 112 was disposed was 100 mm while being fixed and supplied to the electromagnetic proximity sensor 110 at a high frequency of 10 MHz.

Therefore, it is desirable to design the first diameter R1 to be at least 100 mm while using a high frequency of 10 MHz or more as a condition for expanding the sensing range according to the electromagnetic field while ensuring that the penetration depth of the electromagnetic field is at least 300 mm.

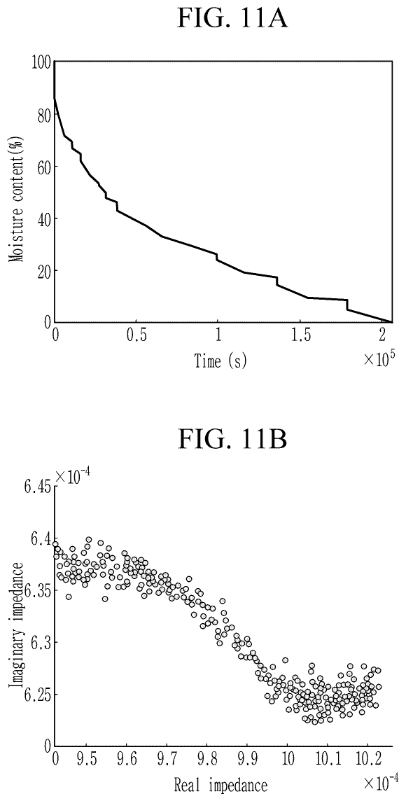

FIGS. 11A and 11B show an experiment for predicting the moisture content in the concrete 1, which is the result of analyzing the detection signal received from the electromagnetic proximity sensor 110 according to the moisture content.

FIG. 11A is a graph illustrating the change in the moisture content in the concrete 1 over time, and FIG. 11B is a graph illustrating the result of an electromagnetic field (impedance) according to the moisture content.

As a result, from the results of FIG. 11A and FIG. 11B having a similar flow, it can be confirmed that the moisture content in the concrete 1 can be predicted through the signal (impedance value) received from the electromagnetic proximity sensor 110.

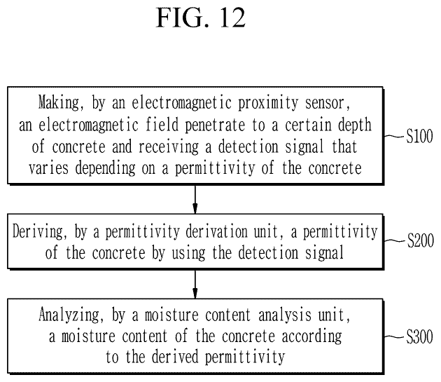

FIGS. 12 to 14 are flowcharts illustrating a concrete moisture content diagnosis method according to an embodiment.

First, as illustrated in FIG. 12, the concrete moisture content diagnosis method according to the present disclosure may include making, by the electromagnetic proximity sensor 110, the electromagnetic field penetrate to a predetermined depth of the concrete 1 and receiving a detection signal that differs according to a permittivity of the concrete 1 (S100), deriving, by the permittivity derivation unit 132, the permittivity of the concrete 1 by using the detection signal (S200), and analyzing, by the moisture content analysis unit 134, the moisture content of the concrete 1 according to the derived permittivity (S300).

The detection signal received by the electromagnetic proximity sensor 110 may be an impedance value for an induced current flowing through the second coil 114 of the electromagnetic proximity sensor 110. The derivation unit 132 may include, for example, hardware, such as a processor and memory device, as well as software, such as computer program code, configured to determine the permittivity based on the detection signal. For example, an equation may be used to determine the permittivity based on the detection signal, or a lookup table may be used that stores different permittivities based on different levels of the detection signal and various other factors, such as coil diameter, current frequency, and other factors. The moisture content analysis unit 134 may include, for example, hardware, such as a processor and memory device, as well as software, such as computer program code, configured to correlate a moisture content with a derived permittivity, for example, based on one or more equations or lookup tables. In addition to the moisture content (e.g., moisture amount), a location of the moisture (e.g., a distance that it has penetrated the concrete) may be determined, again based on one or more equations or data values stored in a lookup table.

The location of the moisture that has penetrated into the concrete 1 may be determined using equations, sensor measurement methods, or the like. However, it is to be understood that the method of determining the moisture location is not limited to these methods, and various other techniques may also be used.

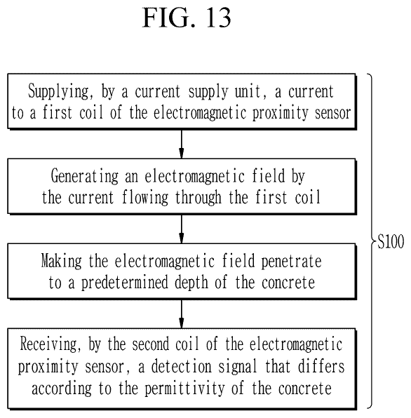

Referring to FIG. 13, the operation S100 in which the electromagnetic proximity sensor 110 receives the detection signal may include supplying, by the current supply unit 120, current to the first coil 112 of the electromagnetic proximity sensor 110, generating an electromagnetic field by the current flowing through the first coil 112, making the electromagnetic field penetrating to a predetermined depth of the concrete 1, and receiving, by the second coil 114 of the electromagnetic proximity sensor 110, a detection signal that differs according to the permittivity of the concrete 1.

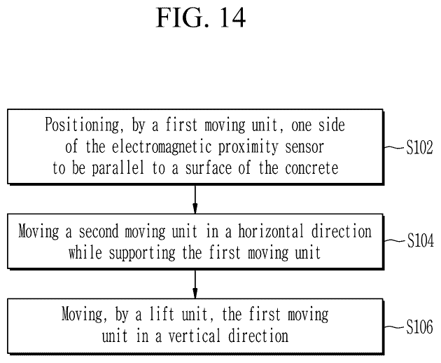

Referring to FIG. 14, the concrete moisture content diagnosis method according to the present disclosure may further include positioning, by the articulated arm 212 of the first moving unit 210, one side of the electromagnetic proximity sensor 110 to be parallel to the surface of the concrete 1 (S102), moving, by the second moving unit 220, in the horizontal direction while supporting the first moving unit 210 (S104), and moving, by the lift unit 230 disposed between the first moving unit 210 and the second moving unit 220, the first moving unit 210 in the vertical direction (S106).

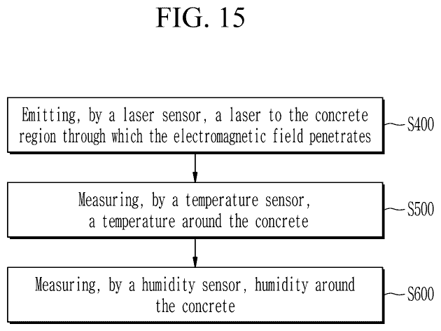

Further, referring to FIG. 15, the concrete moisture content diagnosis method according to the present disclosure may further include emitting, by the laser sensor 240, the area of the concrete 1 where the electromagnetic field penetrates (S400), measuring, by the temperature sensor 250, a temperature around the concrete 1 S500, and measuring, by the humidity sensor 260, the humidity around the concrete 1 S600.

The laser sensor 240 may measure a separation distance between the outer wall of the concrete 1 and one side of the electromagnetic proximity sensor 110. By using the measured distance, the value may be partially corrected so that an error is minimized in the process of analyzing the impedance value.

The temperature sensor 250 and the humidity sensor 260 may measure ambient temperature and humidity, respectively.

The temperature sensor 250 and the humidity sensor 260 may partially correct the value so that an error is minimized in the process of analyzing the impedance value in consideration of the electrical resistance value that changes according to the measured temperature and humidity.

As described above, according to the concrete moisture content diagnosis device 100, the system 10, and the diagnosis method using the same, the location and degree of leakage inside the concrete can be diagnosed with high accuracy by analyzing the concrete moisture content by using a detection signal that differs according to the permittivity of concrete. For example, different resulting detection signals can indicate different degrees of leakage (e.g., different total moisture content within a paritcular volume or segment of concrete) as well as locations of leakage (e.g., distance into the concrete that moisture has penetrated).

The location of the leakage may be determined by analyzing the moisture content within the concrete 1. As the electromagnetic proximity sensor 110 moves along the region of the concrete 1 to be diagnosed, the detection signal may be used to generate a graphical representation indicating the position and penetration depth of the moisture within each region of the concrete 1.

In particular, it is significant in that it is possible to check the progress of the leak before the leak is exposed to the outer wall of the concrete and track the location of the leak inside the concrete.

Furthermore, the moisture content diagnosis device includes the autonomous moving device so that the moisture content diagnosis device is capable of performing diagnosis while moving along the outer wall of concrete, so that multi-faceted diagnosis is possible without being affected by the user's skill level and without being limited to the height of the concrete structure.

Although the embodiments of the present disclosure have been described, the present disclosure is not limited thereto, and it is possible to carry out various modifications within the scope of the claims, the detailed description of the invention, and the accompanying drawings, and the modifications belong to the scope of the present disclosure as a matter of course.

What is claimed is:

1. A device for diagnosing a moisture content of concrete, the device comprising:
an electromagnetic proximity sensor configured to cause an electromagnetic field to penetrate to a certain depth of concrete and to receive a detection signal that varies depending on a permittivity of the concrete within the covered region of the electromagnetic field;
a current supply unit configured to supply current to the electromagnetic proximity sensor; and
a diagnostic unit configured to analyze a moisture content of the concrete by using the detection signal.

2. The device of claim 1, wherein:
the diagnostic unit includes:
a permittivity derivation unit configured to derive the permittivity of the concrete by using the detection signal; and
a moisture content analysis unit configured to analyze the moisture content of the concrete according to the derived permittivity.

3. The device of claim 1, wherein:
the electromagnetic proximity sensor includes:
a first coil, which has a shape wound to have a first diameter, and through which the supplied current flows;
a second coil, which is spaced apart from an inside of the first coil and has a shape wound to have a second diameter smaller than the first diameter, and through which an induction current flows; and
a support structure configured to support the shapes of the first coil and the second coil.

4. The device of claim 3, wherein:
the first diameter is 80 mm or more.

5. The device of claim 3, wherein:
the support structure includes:
a first support unit, which has a hollow cylindrical shape and is wound with the first coil on an exterior side; and
a second support unit, which has a structure inserted into the first support unit, and around which the second coil is wound on an exterior side.

6. The device of claim 5, wherein:
the support structure further includes:
a plate connected to one side of the first support unit and the second support unit to support the shape of the electromagnetic proximity sensor.

7. The device of claim 1, further comprising:
a current controller configured to control an intensity of the electromagnetic field by controlling the supplied current.

8. The device of claim 1, wherein:
the current supply unit is configured to supply current having a high frequency.

9. A system for diagnosing a moisture content of concrete, the system comprising:
a concrete moisture content diagnosis device which includes an electromagnetic proximity sensor having a first side configured to face the concrete while making an electromagnetic field penetrate into the concrete, and which analyzes a moisture content of the concrete by using a detection signal that varies according to a permittivity of the concrete; and
a moving device configured to connect to a second side of the concrete moisture content diagnosis device to move the concrete moisture content diagnosis device.

10. The system of claim 9, wherein:
the moving device includes:
a first moving unit connected to the second side of the concrete moisture content diagnosis device; and
a second moving unit configured to move in a horizontal direction while supporting the first moving unit.

11. The system of claim 10, wherein:
the first moving unit has an articulated structure.

12. The system of claim 11, wherein:
the articulated structure is an arm that has a plurality of joints and segments, and
the arm is rotatable based on the plurality of joints and segments.

13. The system of claim 10, further comprising:
a lift disposed between the first moving unit and the second moving unit,
wherein the lift moves the first moving unit in a vertical direction.

14. The system of claim 9, wherein:
the moving device further includes a laser sensor configured to emit a laser, and
the laser sensor is configured to emit a laser to the concrete area where the electromagnetic field penetrates.

15. The system of claim 9, wherein:
the moving device further includes:
a temperature sensor configured to measure a temperature around the concrete; and
a humidity sensor configured to measure humidity around the concrete.

16. The system of claim 9, wherein:
the electromagnetic proximity sensor is configured to analyze the moisture content of the concrete in a non-contact state with the concrete.

17. A method of diagnosing a moisture content of concrete, the method comprising:
making, by an electromagnetic proximity sensor, an electromagnetic field penetrate to a certain depth of concrete and receiving a detection signal that varies depending on a permittivity of the concrete;
deriving, a permittivity of the concrete by using the detection signal; and
determining a moisture content of the concrete according to the derived permittivity.

18. The method of claim 17, wherein:
the receiving of the detection signal by the electromagnetic proximity sensor includes:
supplying, by a current supply unit, a current to a first coil of the electromagnetic proximity sensor;
generating an electromagnetic field by the current flowing through the first coil;
making the electromagnetic field penetrate to a predetermined depth of the concrete; and
receiving, by a second coil of the electromagnetic proximity sensor, a detection signal that varies according to the permittivity of the concrete.

19. The method of claim 17, further comprising:
positioning, by a first moving unit, one side of the electromagnetic proximity sensor to be parallel to a surface of the concrete;
moving a second moving unit in a horizontal direction while supporting the first moving unit; and
moving, by a lift disposed between the first moving unit and the second moving unit, the first moving unit in a vertical direction.

20. The method of claim 17, further comprising:
emitting, by a laser sensor, a laser to the concrete region through which the electromagnetic field penetrates;
measuring, by a temperature sensor, a temperature around the concrete; and
measuring, by a humidity sensor, humidity around the concrete.
